(19) United States
(12) Patent Application Publication
(10) Pub. No.: US 2026/0324704 A1
(43) Pub. Date: Oct. 22, 2026

(54) MOISTURE-PROOF ANTI-CAKING A-LIPOIC ACID

(71) Applicant: NANJING NUTRABUILDING BIO-TECH CO., LTD., Nanjing (CN)

(72) Inventors: Chengfei WANG, Nanjing (CN); Wei WANG, Nanjing (CN); Shawn WELLS, Frisco, TX (US); Kylin LIAO, Nanjing (CN)

(21) Appl. No.: 19/677,882

(22) Filed: May 14, 2026

Related U.S. Application Data
(63) Continuation of application No. PCT/CN2024/131930, filed on Nov. 14, 2024.

(30) Foreign Application Priority Data
Nov. 16, 2023 (WO) PCT/CN2023/132008

(51) Int. Cl. A23P 10/43; A23K 20/121; A23K 20/163; A23K 20/28; C07D 339/04

(52) U.S. Cl. CPC A23P 10/43; A23K 20/121; A23K 20/163; A23K 20/28; C07D 339/04

(57) ABSTRACT

The invention provides a moisture-proof anti-caking α-lipoic acid and a preparation method thereof. Through the auxiliary material selection and preparation method treatment of raw materials, the problems of easy hardening of α-lipoic acid are solved, the stability of α-lipoic acid is improved, and at the same time, the requirement of high content is met, and great convenience is brought to transportation, storage and application scenes, and high applicability is brought to users.

### CROSS REFERENCE TO RELATED APPLICATION

This application is a continuation-in-part application of International Patent Application No. PCT/CN2024/131930, filed on Nov. 14, 2024, which claims the priority of the International Application No. PCT/CN2023/132008, filed on Nov. 16, 2023, the contents of all of which are incorporated herein by reference in their entirety.

### FIELD OF THE INVENTION

The invention belongs to the technical field of dietary supplements and/or preparations; and particularly relates to a moisture-proof anti-caking α-lipoic acid and a preparation method thereof.

### BACKGROUND OF THE INVENTION

Alpha-lipoic acid (α-lipoic acid) is a coenzyme existing in mitochondria, similar to vitamins, which can eliminate free radicals that lead to accelerated aging and disease. α-lipoic acid is absorbed by intestinal tract in the body and enters cells, which has the characteristics of both fat solubility and water solubility. As a coenzyme, α-lipoic acid plays a role in two key oxidative decarboxylation reactions, namely, in pyruvate dehydrogenase complex and α-ketoglutarate dehydrogenase complex, it catalyzes the generation and transfer of acyl groups. α-lipoic acid can accept acyl group, such as acetyl group from pyruvate to form a thioester bond, and then transfer the acetyl group to the sulfur atom of a coenzyme A molecule. The dihydrothioctamide forming prosthetic group can be oxidized by dihydrothioctamide dehydrogenase (requiring NAD⁺) to regenerate oxidized thioctamide. α-lipoic acid has a disulfide five-membered ring structure, high electron density, obvious electrophilicity and the ability to react with free radicals, so it has oxidation resistance, high health care function and medical value (such as anti-fatty liver and lowering plasma cholesterol). In addition, the sulfhydryl group of α-lipoic acid is easy to undergo redox reaction, so it can protect sulfhydryl enzyme from heavy metal ions. α-lipoic acid is widely distributed in nature, especially in liver and yeast cells. α-lipoic acid often exists with vitamin B1 in food. In addition, it also has the functions of restoring fatigue and improving dementia. Therefore, α-lipoic acid has important research value in the field of health care.

α-lipoic acid has a molecular formula of C₈H₁₄O₂S₂ and a relative molecular weight of 206.3, which is slightly soluble in water and easily soluble in ethanol. It often absorbs moisture and hardens during transportation, storage and application, which brings a lot of inconvenience. Therefore, it is urgent to develop a moisture-proof anti-caking α-lipoic acid product to solve the problems of moisture absorption and hardening, and this product will have a very broad market prospect.

### SUMMARY OF THE INVENTION

In order to solve the above problems, the present invention provides a moisture-proof anti-caking α-lipoic acid and a preparation method thereof. Through the auxiliary material selection and preparation method treatment of raw materials, the problems of easy moisture absorption and hardening of α-lipoic acid are solved, the stability of α-lipoic acid is improved, and at the same time, the requirement of high content is met, and great convenience is brought to transportation, storage and application scenes, and high applicability is brought to users. In addition, the preparation process is simple and the production cost is low.

In one aspect, the invention provides a moisture-proof anti-caking α-lipoic acid, wherein the moisture-proof anti-caking α-lipoic acid comprises, by weight, not less than 20% of α-lipoic acid.

In some embodiments, the moisture-proof anti-caking α-lipoic acid comprises, by weight, not less than 25%, 30%, 35%, 40%, 45%, 50%, 55%, 60% of α-lipoic acid. In some embodiments, the moisture-proof anti-caking α-lipoic acid comprises, by weight, not less than 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95%, 97%, 99% of α-lipoic acid.

In some embodiments, the moisture-proof anti-caking α-lipoic acid comprises, by weight, 10-99%, 15-98%, 20-97%, 25-96%, 30-95%, 35-94%, 40-93%, 45-92%, 50-90% of α-lipoic acid. In some embodiments, the moisture-proof anti-caking α-lipoic acid comprises, by weight, 20-99%, 21-98%, 22-97%, 25-96%, 28-95%, 30-95%, 35-92%, 40-90% of α-lipoic acid.

In some embodiments, the moisture-proof anti-caking α-lipoic acid comprises, by weight, 40-98% of α-lipoic acid; 1-40% of one or more auxiliary materials selected from microcrystalline cellulose, compressible starch, lactose or mannitol; 1-10% of one or more auxiliary materials selected from silicon dioxide, magnesium stearate, calcium hydrogen phosphate, calcium silicate, micro-powder silica gel.

In some embodiments, the moisture-proof anti-caking α-lipoic acid comprises, by weight, 50-95% of α-lipoic acid; 2-40% of one or more auxiliary materials selected from microcrystalline cellulose, mannitol; 2-8% of silicon dioxide.

In some embodiments, the moisture-proof anti-caking α-lipoic acid comprises, by weight, 60-90% of α-lipoic acid; 2-33% of one or more auxiliary materials selected from microcrystalline cellulose, mannitol; 2-5% of silicon dioxide. In some embodiments, the moisture-proof anti-caking α-lipoic acid comprises, by weight, 60-90% of α-lipoic acid; 7-33% of one or more auxiliary materials selected from microcrystalline cellulose, mannitol; 2-5% of silicon dioxide.

In some embodiments, the moisture-proof anti-caking α-lipoic acid further comprises, by weight, 1-8% of one or more auxiliary materials selected from hydroxypropyl methylcellulose, maltodextrin, Arabic gum. In some embodiments, the moisture-proof anti-caking α-lipoic acid further comprises, by weight, 1-2% of one or more auxiliary materials selected from hydroxypropyl methylcellulose, maltodextrin, Arabic gum. In some embodiments, the moisture-proof anti-caking α-lipoic acid further comprises, by weight, 1-2% of hydroxypropyl methylcellulose. In some embodiments, the moisture-proof anti-caking α-lipoic acid may further comprise, by weight, 1-3% of ethyl cellulose.

In some embodiments, the moisture-proof anti-caking α-lipoic acid is in granular or powdery form.

In some embodiments, the particle size of the moisture-proof anti-caking α-lipoic acid is 10-100 meshes. In some embodiments, the particle size of the moisture-proof anti-caking α-lipoic acid is 20-80 meshes.

In some embodiments, the moisture-proof anti-caking α-lipoic acid is formulated into nutritional supplement, food, beverage or animal feed.

In some embodiments, the moisture-proof anti-caking α-lipoic acid is prepared into solid preparation or liquid preparation.

In some embodiments, the passing rate of the moisture-proof anti-caking α-lipoic acid through a 24-mesh sieve is not less than 50%, 80%, or 85% after standing for a period of time. In some embodiments, the passing rate of the moisture-proof anti-caking α-lipoic acid through a 24-mesh sieve is not less than 80%, 85%, 87% after standing for 0.5 hours. In some embodiments, the passing rate of the moisture-proof anti-caking α-lipoic acid through a 24-mesh sieve is not less than 75%, 78%, 80% after standing for 1.5 hours. In some embodiments, the passing rate of the moisture-proof anti-caking α-lipoic acid through a 24-mesh sieve is not less than 45%, 50%, 55% after standing for 7 hours.

In some embodiments, the moisture-proof anti-caking α-lipoic acid is placed under high humidity conditions, e.g., at temperature of 40° C.±2° C. and relative humidity of 75%+5%.

In some embodiments, the repose angle of the moisture-proof anti-caking α-lipoic acid is sustainably unchanged or changes not more than 6°, 8°, 10°, 12° after standing for a period of time. In some embodiments, the repose angle of the moisture-proof anti-caking α-lipoic acid is sustainably unchanged or changes not more than 6°, 8°, 10° after standing at 40° C.±2° C. and 75%+5% for 3 h. In some embodiments, the repose angle of the moisture-proof anti-caking α-lipoic acid is sustainably unchanged or changes not more than 8°, 10°, 12° after standing at 40° C.±2° C. and 75%+5% for 7 h. In some embodiments, the repose angle of the moisture-proof anti-caking α-lipoic acid is not higher than 42°, 44°, 46°, 48°. In some embodiments, the repose angle of the moisture-proof anti-caking α-lipoic acid is not higher than 46°, 48°, 50°, 52° after standing for a period of time. In some embodiments, the repose angle of the moisture-proof anti-caking α-lipoic acid is not higher than 46°, 48°, 50° after standing at 40° C.±2° C. and 75%+5% for 3 h. In some embodiments, the repose angle of the moisture-proof anti-caking α-lipoic acid is not higher than 48°, 50°, 52° after standing at 40° C.±2° C. and 75%=5% for 7 h.

In another aspect, the invention provides a method for preparing moisture-proof anti-caking α-lipoic acid, which comprises mixing the following components of prescribed amounts: α-lipoic acid; one or more auxiliary materials selected from microcrystalline cellulose, compressible starch, lactose or mannitol; one or more auxiliary materials selected from silicon dioxide, magnesium stearate, calcium hydrogen phosphate, calcium silicate, micro-powder silica gel; granulating; to obtain the moisture-proof anti-caking α-lipoic acid.

In some embodiments, the moisture-proof anti-caking α-lipoic acid comprises, by weight, 60-90% of α-lipoic acid; 2-33% of one or more auxiliary materials selected from microcrystalline cellulose, mannitol; 2-5% of silicon dioxide. In some embodiments, the moisture-proof anti-caking α-lipoic acid comprises, by weight, 60-90% of α-lipoic acid; 7-33% of one or more auxiliary materials selected from microcrystalline cellulose, mannitol; 2-5% of silicon dioxide.

In some embodiments, mixing further comprises mixing one or more auxiliary materials selected from hydroxypropyl methylcellulose, maltodextrin, Arabic gum of prescribed amounts. In some embodiments, mixing further comprises mixing hydroxypropyl methylcellulose of a prescribed amount. In some embodiments, mixing further comprises mixing ethyl cellulose of a prescribed amount.

In some embodiments, the moisture-proof anti-caking α-lipoic acid comprises, by weight, 1-8% of one or more auxiliary materials selected from hydroxypropyl methylcellulose, maltodextrin, Arabic gum. In some embodiments, the moisture-proof anti-caking α-lipoic acid comprises, by weight, 1-2% of one or more auxiliary materials selected from hydroxypropyl methylcellulose, maltodextrin, Arabic gum. In some embodiments, the moisture-proof anti-caking α-lipoic acid comprises, by weight, 1-2% of hydroxypropyl methylcellulose. In some embodiments, the moisture-proof anti-caking α-lipoic acid comprises, by weight, 1-3% of ethyl cellulose.

In some embodiments, the granulation is dry granulation.

In some embodiments, the moisture-proof anti-caking α-lipoic acid prepared by the method is used for preparing food, beverage, nutrition, skin care products or cosmetics.

This summary is provided to introduce the selection of concepts in a simplified form, which are further described in the detailed description below. Summary is not intended to identify key features or essential features of the claimed subject matter, nor is it intended to be used to limit the scope of the claimed subject matter.

### DETAILED DESCRIPTION OF THE INVENTION

Reference will now be made in detail to the preferred embodiments of the present disclosure, examples of which are further illustrated. While the present disclosure will be described in conjunction with the preferred embodiments, it will be understood that they are not intended to limit the present disclosure to these embodiments. To the contrary, the present disclosure is intended to cover alternatives, modifications and equivalents, which may be included within the spirit and scope of the present disclosure as defined by the claims.

As used herein, the term “or” is meant to include both “and” and “or.” In other words, the term “or” may also be replaced with “and/or.”

As used herein, the singular forms “a,” “an” and “the” are intended to include the plural forms as well, unless the context clearly indicates otherwise.

As used herein, the term “comprise” or “include” or their conjugations, refer to a situation where said terms are used in their non-limiting sense to mean that items following the word are included, but items not specifically mentioned are not excluded. It also encompasses the more limiting verb ‘to consist essentially of’ and ‘to consist of’.

### EXAMPLES

In the present disclosure, unless otherwise specified, scientific and technical terms used herein have the meanings commonly understood by those skilled in the art. Moreover, the laboratory operation steps used herein are all routine steps widely used in the corresponding fields.

The raw materials or reagents used in the present disclosure are commercially available unless otherwise specified. All reagents are commercial grade and used according to the received standards.

The following examples are illustrative of selected embodiments of the present disclosure and are not meant to limit the scope of the present disclosure.

### Example 1: Preparation of Moisture-Proof Anti-Caking α-Lipoic Acid

A moisture-proof anti-caking α-lipoic acid comprises the following components in percentage by weight:

| | |
|---|---|
| α-lipoic acid | 80% |
| microcrystalline cellulose | 15% |
| silicon dioxide | 5%. |

α-lipoic acid was sieved with a 30-mesh sieve, α-lipoic acid, microcrystalline cellulose and silicon dioxide of prescribed amounts were weighed and put into a three-dimensional mixer, dry granulation was carried out, to obtain the moisture-proof anti-caking α-lipoic acid.

### Example 2: Preparation of Moisture-Proof Anti-Caking α-Lipoic Acid

A moisture-proof anti-caking α-lipoic acid comprises the following components in percentage by weight:

| | |
|---|---|
| α-lipoic acid | 80% |
| mannitol | 10% |
| microcrystalline cellulose | 5% |
| silicon dioxide | 5%. |

α-lipoic acid, mannitol was each sieved with a 30-mesh sieve, α-lipoic acid, mannitol, microcrystalline cellulose and silicon dioxide of prescribed amounts were weighed and put into a three-dimensional mixer, dry granulation was carried out, to obtain the moisture-proof anti-caking α-lipoic acid.

### Example 3: Preparation of Moisture-Proof Anti-Caking α-Lipoic Acid

A moisture-proof anti-caking α-lipoic acid comprises the following components in percentage by weight:

| | |
|---|---|
| α-lipoic acid | 80% |
| mannitol | 9% |
| microcrystalline cellulose | 4% |
| hydroxypropyl methylcellulose | 2% |
| silicon dioxide | 5%. |

α-lipoic acid, mannitol was each sieved with a 30-mesh sieve, α-lipoic acid, mannitol, microcrystalline cellulose, hydroxypropyl methylcellulose, and silicon dioxide of prescribed amounts were weighed and put into a three-dimensional mixer, dry granulation was carried out, to obtain the moisture-proof anti-caking α-lipoic acid.

### Example 4: Preparation of Moisture-Proof Anti-Caking α-Lipoic Acid

A moisture-proof anti-caking α-lipoic acid comprises the following components in percentage by weight:

| | |
|---|---|
| α-lipoic acid | 90% |
| mannitol | 5% |
| microcrystalline cellulose | 2% |
| hydroxypropyl methylcellulose | 1% |
| silicon dioxide | 2%. |

α-lipoic acid, mannitol was each sieved with a 30-mesh sieve, α-lipoic acid, mannitol, microcrystalline cellulose, hydroxypropyl methylcellulose, and silicon dioxide of prescribed amounts were weighed and put into a three-dimensional mixer, dry granulation was carried out, to obtain the moisture-proof anti-caking α-lipoic acid.

### Example 5: Preparation of Moisture-Proof Anti-Caking α-Lipoic Acid

A moisture-proof anti-caking α-lipoic acid comprises the following components in percentage by weight:

| | |
|---|---|
| α-lipoic acid | 70% |
| mannitol | 15% |
| microcrystalline cellulose | 8% |
| hydroxypropyl methylcellulose | 2% |
| silicon dioxide | 5%. |

α-lipoic acid, mannitol was each sieved with a 30-mesh sieve, α-lipoic acid, mannitol, microcrystalline cellulose, hydroxypropyl methylcellulose, and silicon dioxide of prescribed amounts were weighed and put into a three-dimensional mixer, dry granulation was carried out, to obtain the moisture-proof anti-caking α-lipoic acid.

### Example 6: Preparation of Moisture-Proof Anti-Caking α-Lipoic Acid

A moisture-proof anti-caking α-lipoic acid comprises the following components in percentage by weight:

| | |
|---|---|
| α-lipoic acid | 60% |
| mannitol | 20% |
| microcrystalline cellulose | 13% |
| hydroxypropyl methylcellulose | 2% |
| silicon dioxide | 5%. |

α-lipoic acid, mannitol was each sieved with a 30-mesh sieve, α-lipoic acid, mannitol, microcrystalline cellulose, hydroxypropyl methylcellulose, and silicon dioxide of prescribed amounts were weighed and put into a three-dimensional mixer, dry granulation was carried out, to obtain the moisture-proof anti-caking α-lipoic acid.

### Example 7: Investigation on Moisture-Proof and Anti-Caking of Moisture-Proof Anti-Caking α-Lipoic Acid Samples

Specific test method: A proper amount of test sample was weighed and put in a dry glass bottle (with an outer diameter of 50 mm and a height of 100 mm) opening, placed in a box with a temperature of 40° C.±2° C. and a relative humidity of 75%±5%. At regular intervals (at 0.5 h, 1 h, 1.5 h, 2 h, 3 h, 5 h and 7 h respectively), the glass bottle was taken out and the passing rate of the sample through a 24-mesh sieve was detected. The passing rate was calculated according to the formula:

$$passing rate = 100 \% \times \frac{weight of material passing through sieve}{total weight of material} .$$

The passing rate results of moisture-proof and anti-caking α-lipoic acid samples are shown in Table 1.

TABLE 1

| Sample | 0 h | 0.5 h | 1 h | 1.5 h | 2 h | 3 h | 5 h | 7 h |
|---|---|---|---|---|---|---|---|---|
| Raw material | 100.0% | 32.4% | 10.3% | 1.9% | 1.3% | 0.7% | 0.3% | 0.0% |
| Example 1 | 100.0% | 89.7% | 87.1% | 83.2% | 81.7% | 81.0% | 75.4% | 70.3% |
| Example 2 | 100.0% | 97.1% | 95.9% | 94.7% | 94.0% | 93.6% | 92.1% | 85.4% |
| Example 3 | 100.0% | 98.9% | 97.3% | 96.4% | 95.1% | 92.7% | 91.4% | 89.0% |
| Example 4 | 100.0% | 87.1% | 83.1% | 80.1% | 75.2% | 71.9% | 67.2% | 57.1% |
| Example 5 | 100.0% | 99.1% | 98.7% | 97.2% | 96.4% | 94.9% | 93.2% | 91.7% |
| Example 6 | 100.0% | 99.6% | 99.3% | 98.2% | 97.3% | 96.0% | 95.2% | 93.2% |

As can be seen from Table 1, compared with raw material, the moisture-proof and anti-caking performance of moisture-proof anti-caking α-lipoic acid samples of various examples of the present invention is obviously improved under the conditions of temperature of 40° C.±2° C. and relative humidity of 75%±5%, while having a high load of not less than 60%; and samples of some examples are particularly excellent, and they have excellent moisture-proof and anti-caking effects and good stability under the temperature and humidity conditions of accelerated experiments. For example, the sample of Example 3, which was obtained by mixing mannitol, microcrystalline cellulose and other auxiliary materials and dry granulation, has a loading capacity of as high as 80%; besides, passing rate remains close to 90% after being placed (standing) at 40° C.±2° C. and 75%±5% relative humidity for 7 hours. It has excellent stability and fluidity, and has excellent moisture-proof and anti-caking effects. The passing rate of the samples of the invention through a 24-mesh sieve is not less than 85% after standing at 40° C.±2° C. and 75%±5% relative humidity for 0.5 hours; not less than 80% after standing at 40° C.±2° C. and 75%±5% relative humidity for 1.5 hours; not less than 50% after standing at 40° C.±2° C. and 75%±5% relative humidity for 7 hours. After being placed (standing), the moisture-proof anti-caking α-lipoic acid samples of the invention are obviously not easy to absorb moisture and harden in appearance, and the fluidity and stability are obviously improved.

The repose angle of each sample (before standing, after standing at 40° C.±2° C. and 75%±5% for 3 h, 7 h) was measured by using the fixed funnel injection method according to the following steps: (1) Apparatus preparation: Vertically fix the funnel above a flat circular disk, and adjust the exact height so that the diameter of the resulting powder cone can be controlled. (2) Adding material: Block the funnel outlet with a stopper rod, pour a sufficient amount of sample into the funnel, then withdraw the stopper rod, allowing the powder to fall freely onto the center of the disk to form a symmetrical cone. (3) Powder accumulation treatment: Gently tap the funnel wall or lightly stir with a thin rod, without directly vibrating or impacting the disk. Once the material has completely ceased flowing and the cone has stabilized, the measurement is carried out. (4) Use a protractor to directly measure the angle between the cone slope and the horizontal plane, or measure the cone height (h) and base radius (r) and calculate the angle. The results are shown in Table 2.

TABLE 2

| Sample | 0 h | 3 h | 7 h |
|---|---|---|---|
| Raw material | 54° | unmeasurable | unmeasurable |
| Example 1 | 42° | 46° | 48° |
| Example 2 | 40° | 42° | 46° |
| Example 3 | 39° | 42° | 45° |
| Example 4 | 40° | 43° | 46° |
| Example 5 | 38° | 39° | 42° |
| Example 6 | 37° | 39° | 42° |

The repose angle of the moisture-proof anti-caking α-lipoic acid is sustainably unchanged or changes not more than 6°, 8°, 10°, 12° after standing for a period of time. The repose angle of the moisture-proof anti-caking α-lipoic acid is sustainably unchanged or changes not more than 6°, 8°, 10° after standing at 40° C.±2° C. and 75%±5% for 3 h. The repose angle of the moisture-proof anti-caking α-lipoic acid is sustainably unchanged or changes not more than 8°, 10°, 12° after standing at 40° C.±2° C. and 75%=5% for 7 h. The repose angle of the moisture-proof anti-caking α-lipoic acid is not higher than 42°, 44°, 46°, 48°. The repose angle of the moisture-proof anti-caking α-lipoic acid is not higher than 46°, 48°, 50°, 52° after standing for a period of time. The repose angle of the moisture-proof anti-caking α-lipoic acid is not higher than 46°, 48°, 50° after standing at 40° C.±2° C. and 75%±5% for 3 h. The repose angle of the moisture-proof anti-caking α-lipoic acid is not higher than 48°, 50°, 52° after standing at 40° C.±2° C. and 75%±5% for 7 h.

When the moisture-proof anti-caking α-lipoic acid of the present invention comprises not less than 20%, 25%, 30%, 35%, 40%, 45%, 50%, 55%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95%, 97%, 99% by weight of alpha-lipoic acid, or 10-99%, 15-98%, 20-97%, 25-96%, 30-95%, 35-94%, 40-93%, 45-92%, 50-90% by weight of alpha-lipoic acid, it can achieve more obvious moisture-proof and anti-caking effects, better fluidity and stability than raw materials under the conditions of temperature of 40° C.±2° C., relative humidity of 75%±5% and normal temperature storage.

Although specific embodiments and examples of the present disclosure have been illustrated herein, it will be appreciated by those skilled in the art that any modifications and variations can be made without departing from the spirit of the present disclosure. The examples and illustrations above are not intended to limit the scope of the present disclosure. Any combination of embodiments of the present disclosure, along with any obvious their extension or analogs, are within the scope of the present disclosure. Further, it is intended that the present disclosure encompass any arrangement, which is calculated to achieve that same purpose, and all such variations and modifications fall within the scope of the appended claims.

What is claimed is:

1. A moisture-proof anti-caking α-lipoic acid, wherein the moisture-proof anti-caking α-lipoic acid comprises, by weight, not less than 20% of α-lipoic acid.

2. The moisture-proof anti-caking α-lipoic acid of claim 1, wherein the moisture-proof anti-caking α-lipoic acid comprises, by weight, not less than 25%, 30%, 35%, 40%, 45%, 50%, 55%, 60% of α-lipoic acid.

3. The moisture-proof anti-caking α-lipoic acid of claim 1, wherein the moisture-proof anti-caking α-lipoic acid comprises, by weight, 40-98% of α-lipoic acid; 1-40% of one or more auxiliary materials selected from microcrystalline cellulose, compressible starch, lactose or mannitol; 1-10% of one or more auxiliary materials selected from silicon dioxide, magnesium stearate, calcium hydrogen phosphate, calcium silicate, micro-powder silica gel.

4. The moisture-proof anti-caking α-lipoic acid of claim 1, wherein the moisture-proof anti-caking α-lipoic acid comprises, by weight, 50-95% of α-lipoic acid; 2-40% of one or more auxiliary materials selected from microcrystalline cellulose, mannitol; 2-8% of silicon dioxide.

5. The moisture-proof anti-caking α-lipoic acid of claim 1, wherein the moisture-proof anti-caking α-lipoic acid comprises, by weight, 60-90% of α-lipoic acid; 2-33% of one or more auxiliary materials selected from microcrystalline cellulose, mannitol; 2-5% of silicon dioxide.

6. The moisture-proof anti-caking α-lipoic acid of claim 3, wherein the moisture-proof anti-caking α-lipoic acid further comprises, by weight, 1-2% of one or more auxiliary materials selected from hydroxypropyl methylcellulose, maltodextrin, Arabic gum.

7. The moisture-proof anti-caking α-lipoic acid of claim 1, wherein the moisture-proof anti-caking α-lipoic acid comprises, by weight, 60-90% of α-lipoic acid; 7-33% of one or more auxiliary materials selected from microcrystalline cellulose, mannitol; 2-5% of silicon dioxide.

8. The moisture-proof anti-caking α-lipoic acid of claim 7, wherein the moisture-proof anti-caking α-lipoic acid further comprises, by weight, 1-2% of hydroxypropyl methylcellulose.

9. The moisture-proof anti-caking α-lipoic acid of claim 1, wherein the moisture-proof anti-caking α-lipoic acid is in granular or powdery form.

10. The moisture-proof anti-caking α-lipoic acid of claim 9, wherein the particle size of the moisture-proof anti-caking α-lipoic acid is 10-100 meshes.

11. The moisture-proof anti-caking α-lipoic acid of claim 9, wherein the particle size of the moisture-proof anti-caking α-lipoic acid is 20-80 meshes.

12. The moisture-proof anti-caking α-lipoic acid of claim 1, wherein the moisture-proof anti-caking α-lipoic acid is formulated into nutritional supplement, food, beverage or animal feed.

13. The moisture-proof anti-caking α-lipoic acid of claim 1, wherein the moisture-proof anti-caking α-lipoic acid is prepared into solid preparation or liquid preparation.

14. The moisture-proof anti-caking α-lipoic acid of claim 1, wherein the passing rate of the moisture-proof anti-caking α-lipoic acid through a 24-mesh sieve is not less than 85% after standing for 0.5 hours.

15. The moisture-proof anti-caking α-lipoic acid of claim 1, wherein the passing rate of the moisture-proof anti-caking α-lipoic acid through a 24-mesh sieve is not less than 80% after standing for 1.5 hours.

16. The moisture-proof anti-caking α-lipoic acid of claim 1, wherein the passing rate of the moisture-proof anti-caking α-lipoic acid through a 24-mesh sieve is not less than 50% after standing for 7 hours.

17. The moisture-proof anti-caking α-lipoic acid of claim 1, wherein the repose angle of the moisture-proof anti-caking α-lipoic acid is sustainably unchanged or changes not more than 8° after standing for a period of time.

18. The moisture-proof anti-caking α-lipoic acid of claim 1, wherein the repose angle of the moisture-proof anti-caking α-lipoic acid is not higher than 44°.

19. The moisture-proof anti-caking α-lipoic acid of claim 1, wherein the repose angle of the moisture-proof anti-caking α-lipoic acid is not higher than 48° after standing for a period of time.

20. The moisture-proof anti-caking α-lipoic acid of claim 1, wherein the passing rate of the moisture-proof anti-caking α-lipoic acid through a 24-mesh sieve is not less than 50% after standing for a period of time.
